(19) United States
(12) Patent Application Publication
(10) Pub. No.: US 2025/0345478 A1
(43) Pub. Date: Nov. 13, 2025

(54) PHOTOCROSSLINKABLE SYNTHETIC POLYMERS

(71) Applicant: Cook Biotech Incoporated, West Lafayette, IN (US)

(72) Inventors: Sydney E. Hollingshead, Lafayette, IN (US); Tyler A. Novak, Lafayette, IN (US)

(73) Assignee: Cook Biotech Incoporated, West Lafayette, IN (US)

(21) Appl. No.: 18/862,625

(22) PCT Filed: May 5, 2023
(86) PCT No.: PCT/US2023/066681

§ 371 Date: Nov. 4, 2024

Related U.S. Application Data
(60) Provisional application No. 63/338,656, filed on May 5, 2022.
(60) Provisional application No. 63/456,565, filed on Apr. 3, 2023.

(51) Int. Cl. A61L 24/00; A61L 24/04; B01J 31/12; B01J 35/39

(52) U.S. Cl. CPC A61L 24/0031; A61L 24/0042; A61L 24/043; B01J 31/12; B01J 35/39

(57) ABSTRACT

In certain embodiments, the present disclosure provides adhesive compositions and medical graft materials comprising adhesive compositions. In some forms, such adhesive compositions comprise are photocurable and comprise one or more phenol-enriched synthetic polymers.

### CROSS REFERENCE TO RELATED APPLICATION

This application claims the benefit of U.S. Provisional Application No. 63/338,656 filed May 5, 2022, and U.S. Provisional Application No. 63/456,565 filed Apr. 3, 2023, which are hereby incorporated herein by reference in their entirety.

### BACKGROUND

The present disclosure resides generally in the field of medical compositions and in particular aspects to photocurable compositions such as adhesives.

As further background, adhesive compositions and/or photocurable compositions are used in a variety of medical and non-medical applications. For example, when surgical wounds on liquid-containing or gas-containing structures are closed with a suture or staple line, there is a risk of liquid or gas leakage from the closed wound site. Surgical adhesives are applied over the suture and/or staple line to reduce the risk of leakage. In other uses, surgical adhesives and similar compositions can be applied to bond patient tissues to one another and/or bond implant materials to patient tissues, as well as to provide a bulking function to increase tissue volume. While some work has been done in these fields, needs remain for improved and/or alternative compositions and in particular compositions that crosslink when exposed to light.

### SUMMARY

In certain aspects, the present disclosure provides compositions and methods for crosslinking a composition. In accordance with certain embodiments, the present disclosure provides compositions and methods suitable for photocrosslinking a composition. In some forms, such compositions comprise a phenol-enriched synthetic polymer. Accordingly, in one embodiment, the present disclosure provides a photocurable adhesive comprising, a photoactivatable catalyst such as a photoactivatable metal-ligand complex, an electron acceptor, and a phenol-enriched synthetic polymer. In certain embodiments, the synthetic polymer comprises polyethylene glycol, polyvinyl alcohol, polylactic acid, polyglycolic acid, poly(lactic-co-glycolic acid), and/or poly(glycerol sebacate). In certain embodiments, the synthetic polymer has multiple arms and has been chemically modified so that each arm terminates in a phenol group. In accordance with some forms, the synthetic polymer comprises a multi-arm polyethylene glycol, for example a 2-arm polyethylene glycol, 4-arm polyethylene glycol, and/or 8-arm polyethylene glycol. In certain embodiments, the electron acceptor comprises sodium persulfate. In accordance with certain embodiments, the composition further comprises gelatin, preferably a phenol-modified gelatin. In some forms, the phenol-enriched synthetic polymer and the phenol-modified gelatin are present in amounts such that a ratio of phenol groups of the phenol-enriched synthetic polymer and phenol groups of the phenol-modified gelatin is about 1:2. In some forms, 20% to 80% of the total phenol in the composition is provided by the phenol-enriched synthetic polymer.

In another embodiment, the present disclosure provides a method of crosslinking a synthetic polymer, the method comprising irradiating a composition comprising a photoactivatable catalyst such as a photoactivatable metal-ligand complex, an electron acceptor, and a phenol-enriched synthetic polymer to initiate a cross-linking reaction. In certain embodiments of the method, the composition is exposed to visible light for a duration sufficient to initiate a cross-linking reaction, for example at least thirty seconds or at least sixty second. In certain embodiments, the phenol-enriched synthetic polymer comprises polyethylene glycol, polyvinyl alcohol, polylactic acid, polyglycolic acid, poly(lactic-co-glycolic acid), and/or poly(glycerol sebacate). In accordance with some forms, the synthetic polymer comprises 2-arm polyethylene glycol, 4-arm polyethylene glycol, and/or 8-arm polyethylene glycol. In certain embodiments, the electron acceptor comprises sodium persulfate. In accordance with certain embodiments, the composition further comprises gelatin, preferably a phenol-modified gelatin. In some forms, the phenol-enriched synthetic polymer and the phenol-modified gelatin are present in amounts such that a ratio of phenol groups of the phenol-enriched synthetic polymer and phenol groups of the phenol-modified gelatin is about 1:2. In some forms, 20% to 80% of the total phenol groups in the composition is provided by the phenol-enriched synthetic polymer.

In a further embodiment, the present disclosure provides a medical implant comprising a substrate suitable for implantation, and a medical adhesive carried by the substrate, the medical adhesive comprising a photoactivatable catalyst such as a photoactivatable metal-ligand complex, an electron acceptor, and a phenol-enriched synthetic polymer. In certain embodiments, the substrate comprises an extracellular matrix material, for example one or more of the following: submucosa, renal capsule membrane, dermal collagen, dura mater, pericardium, fascia lata, serosa, peritoneum or basement membrane layers, and/or including liver basement membrane. In accordance with some forms, the extracellular matrix material is in sheet form, and wherein the medical adhesive is carried on a first surface of the sheet form extracellular matrix material. In certain embodiments, the medical adhesive is crosslinked. In certain embodiments the medical implant is contained within a sterile package. In certain embodiments, the phenol-enriched synthetic polymer comprises polyethylene glycol, polyvinyl alcohol, polylactic acid, polyglycolic acid, poly(lactic-co-glycolic acid), and/or poly(glycerol sebacate). In certain embodiments, the phenol-enriched synthetic polymer has been chemically modified so that each arm terminates in a phenol group. In accordance with some forms, the phenol-enriched synthetic polymer comprises 2-arm polyethylene glycol, 4-arm polyethylene glycol, and/or 8-arm polyethylene glycol. In certain embodiments, the electron acceptor comprises sodium persulfate. In accordance with certain embodiments, the composition further comprises gelatin, preferably a phenol-modified gelatin. In some forms, the phenol-enriched synthetic polymer and the phenol-modified gelatin are present in amounts such that a ratio of phenol groups of the phenol-enriched synthetic polymer and phenol groups of the phenol-modified gelatin is about 1:2. In some forms, 20% to 80% of the total phenol in the composition is provided by the phenol-enriched synthetic polymer.

In another embodiment, the present disclosure provides a method of preparing a medical adhesive, the method comprising combining a phenol-enriched synthetic polymer, a phenol-modified gelatin, a photoactivatable catalyst such as a photoactivatable metal-ligand complex and, an electron acceptor. In certain embodiments a first composition comprising the phenol-enriched synthetic polymer, the phenol-modified gelatin, and the photoactivatable catalyst is mixed with a second composition comprising the electron acceptor. In certain embodiments, the phenol-enriched synthetic polymer comprises polyethylene glycol, polyvinyl alcohol, polylactic acid, polyglycolic acid, poly(lactic-co-glycolic acid), and/or poly(glycerol sebacate). In certain embodiments, the phenol-enriched synthetic polymer has been chemically modified so that each arm terminates in a phenol group. In accordance with some forms, the phenol-enriched synthetic polymer comprises 2-arm polyethylene glycol, 4-arm polyethylene glycol, and/or 8-arm polyethylene glycol. In certain embodiments, the electron acceptor comprises sodium persulfate. In accordance with certain embodiments, the composition further comprises gelatin, preferably a phenol-modified gelatin. Tn some forms, the phenol-enriched synthetic polymer and the phenol-modified gelatin are present in amounts such that a ratio of phenol groups of the phenol-enriched synthetic polymer and phenol groups of the phenol-modified gelatin is about 1:2. In some forms, 20%/4 to 80% of the total phenol in the composition is provided by the phenol-enriched synthetic polymer.

In yet another embodiment, the present disclosure provides a method of joining and/or sealing tissues in a surgical procedure, the method comprising applying a medical composition as described above to a tissue portion, and irradiating the tissue medical composition to initiate a cross-linking reaction between one or more endogenous proteins and the phenol-enriched synthetic polymer to seal the tissue portion or join the tissue portion to an adjacent tissue portion.

Additional embodiments, as well as features and advantages of embodiments of the invention, will be apparent from the description herein.

### BRIEF DESCRIPTION OF THE DRAWINGS

FIG. 1 is a perspective view of one embodiment of a substrate including a medical adhesive as disclosed herein.

FIG. 2 is graph showing the results the tensile testing of cured adhesive as described in Example 1.

FIG. 3 is a graph showing the results of testing to determine the Young's modulus value of cured material as described herein with varying PEG shape and molecular mass as described in Example 2.

FIG. 4 is a graph showing the results of testing to determine the strength at failure of cured material as described herein with varying PEG shape and molecular mass as described in Example 3.

FIG. 5 is a graph showing the results of testing to determine the stress at failure of cured material as described herein with varying PEG shape and molecular mass as described in Example 4.

FIG. 6 is a graph showing the results of testing to determine the maximum force of end-to-end wound closure with varying cure times as described in Example 5.

FIG. 7 is a graph showing the results of testing maximum load of various adhesive formulations as described in Example 6.

FIG. 8 is a graph showing the results of testing maximum strain of various adhesive formulations as described in Example 6.

FIG. 9 is a graph showing the results of testing to determine the maximum force of various wound closures and adhesive formulations as described in Example 7.

FIG. 10 is a graph showing the results of testing to determine the maximum force of various wound closures and adhesive formulations as described in Example 8.

FIG. 11 is a graph showing the results of testing to determine the extent of polymer modification with varying concentrations of Bolton Hunter reagent as described in Example 9.

### DESCRIPTION OF THE SELECTED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated graft, and further applications of the principles of the disclosure as illustrated therein are herein contemplated as would normally occur to one skilled in the art to which the disclosure relates.

As disclosed above, aspects of the present disclosure relate to novel adhesive compositions and methods of using same. In certain aspects, the disclosure relates to photocurable liquid adhesives comprising a phenol-enriched synthetic polymer and a photoactivatable crosslinking system, such as one including a photoactivatable catalyst and an electron acceptor. In some forms, the photocurable adhesive will also comprise a liquid carrier, preferably an aqueous liquid carrier, such as water or phosphate buffered saline.

The present disclosure provides adhesive compositions that include a phenol-enriched synthetic polymer. It has been discovered that such compositions upon photocuring advantageously form crosslinks between feruloyl groups of separate polymer molecules of the phenol-enriched synthetic polymer (diferuloyl crosslinks). Such diferuloyl crosslinks are, more generally, crosslinks between phenolic groups of separate polymer molecules (“diphenolic crosslinks”). Where the adhesive composition also includes a polymer containing phenolic groups other than the phenol-enriched synthetic polymer, the photocuring may also form diphenolic crosslinks between the phenol-enriched synthetic polymer molecules and molecules of the other polymer containing phenolic groups, as well as between separate polymer molecules of the other polymer containing phenolic groups. As used herein the term “phenolic group” refers to a phenyl ring having a hydroxyl group directly attached to a carbon atom of the phenyl ring. The phenyl ring can optionally have other functional groups attached thereto. For example, a feruloyl group (which has a 4-hydroxy-3-methoxyphenyl group) is a phenolic group as described herein.

In certain embodiments, the present disclosure provides methods of crosslinking a synthetic polymer. Such methods comprise irradiating a composition comprising a photoactivatable catalyst, an electron acceptor, and a phenol-enriched synthetic polymer thereby initiating a cross-linking reaction. The photoactivatable catalyst may, for example, comprise a photoactivatable metal ligand complex and/or riboflavin. In some forms, the irradiating is conducted prior to implantation of a medical graft to form a crosslinked coating on the medical graft. For example, in certain embodiments the irradiating is conducted prior to placing the cross-linked graft into a sterile medical package. However, in certain embodiments the graft may be irradiated shortly before implantation. In such cases, a medical composition as described herein may be applied to a substrate and irradiated prior to implantation. In certain embodiments, the irradiating is performed in situ, for example to close a wound, join tissue, and/or adhere a medical graft material to patient tissue.

The photocurable adhesive will be irradiated with light at a wavelength that activates the photoactivatable catalyst and initiates the covalent crosslinking reaction. Where the photoactivatable catalyst is or includes a photoactivatable metal ligand complex as disclosed herein, preferably ruthenium tris-bipyridyl chloride, irradiation may be performed using white light (i.e. light including wavelengths between about 400 and about 700 nm). In accordance with certain embodiments, the photocurable adhesive composition as described herein is cured by irradiating it for at least 5 seconds, preferably at least 10 seconds, and typically in the range of about 10 seconds to about 180 seconds, more typically in the range of about 15 seconds to about 60 seconds. The cured adhesive material comprises a crosslinked polymeric matrix including the phenol-enhanced synthetic polymer and having covalent feruloyl-feruloyl crosslinks between polymer molecules. The cured adhesive material can further include a photoactivatable catalyst such as a photoactivatable metal-ligand complex and/or an electron acceptor, and can include a reaction product obtained by photocuring a photocurable adhesive composition including a phenol-enhanced synthetic polymer, the photoactivatable catalyst, the electron acceptor, and a liquid medium.

As used herein the term “matrix protein” refers to isolated and purified extracellular matrix proteins. Suitable matrix proteins for use in the medical compositions may be selected from, but not limited to the group consisting of: fibrinogen, fibrin, collagen, keratin, gelatin, fibronectin, serum albumin, elastin, beta-lactoglobulin, glycinin, glutens, gliadins, resilin and/or laminin, or admixtures thereof. Matrix proteins may be isolated from human or animal sources or can be synthetically produced for instance using recombinant techniques. In some forms, matrix proteins are isolated from ECM source tissues as described herein. In some forms, the matrix protein may be denatured to encourage the formation of phenolic cross-links. Denaturation of a protein may be accomplished by raising or lowering the pH of a solution containing the matrix protein, decreasing or increasing the ionic strength of a solution containing the matrix protein, hydrolysis, or in other ways known to a person skilled in the art.

Turning now to a discussion of synthetic polymers, which may be included in medical compositions as described herein, exemplary synthetic polymers include phenol-containing polymers, such as polyacrylamide and/or polyacrylic acid. Alternative embodiments may include a biodegradable polymer additive such as one or more of: polyethylene glycol, polyvinyl alcohol, polylactic acid, polyglycolic acid, polylactic-co-glycolic acid, and/or polyglycerol sebacate.

In certain embodiments, the synthetic polymer and/or matrix protein is phenol-enriched to render the synthetic polymer and/or matrix protein more susceptible to cross-linking compared to its native state. In accordance with some forms, the medical adhesive comprises a phenol-modified matrix protein, which has been chemically modified to render the matrix protein more susceptible to cross-linking compared to its native state. Such chemical modification may include the modification of amino acid side chains to include of aromatic moieties, for example, amine terminated polyethylene glycol arm(s). By way of example primary amines such as the lysine residues in a protein or synthetic polymer may be modified under mild conditions with Bolton-Hunter reagent (N-succinimidyl-3-[4hydroxyphenyl]propionate) or water-soluble Bolton-Hunter reagent (sulfosuccinimidyl-3-[4-hydroxyphenyl]propionate). Such modification may involve modification of the protein or synthetic polymer to alter its secondary, tertiary or quaternary structure. Additional reagents may be employed to effect sulfhydryl reduction, addition of sulfhydryl or amino groups, protein acylation, etc. In certain forms, the compositions of the present disclosure will include a mixture of an amount of a matrix protein (especially collagen, gelatin or a collagen peptide composition) and/or synthetic polymer with an amount of the corresponding phenol-enriched protein and/or phenol-enriched synthetic polymer. For example, the photocurable adhesive may include the parent (unmodified) matrix protein or synthetic polymer and the corresponding phenol-enriched matrix protein or synthetic polymer in a dry weight ratio in the range of about 1:10 to about 10:1, or about 1:5 to about 5:1, and in some forms about 5:1 to about 2:1.

Certain embodiments comprise polyethylene glycol. Polyethylene glycol may be provided in various geometries and molecular weights. It within the scope of the disclosure to provide medical adhesive compositions comprising various geometries of polyethylene glycol including, linear, branched, star-shaped, Y-shaped, and/or comb shaped. Certain embodiments utilize polyethylene glycol of various molecular weights as well, including polyethylene glycol having a molecular weight of between 400 Da to 40,000 Da, preferably 1,000 Da to 20,000 Da, even more preferably 2,000 Da to 10,000 Da. Certain embodiments comprise polyethylene glycol having a molecular weight of 2,000 Da, 5,000 Da, or 10,000 Da. In accordance with certain embodiments, star shaped, or multi-armed polyethylene glycol is preferred. For example, in some forms the compositions described herein comprise polyethylene glycol having at least 2-arms, at least 4-arms, at least 6-arms, or at least 8-arms. Certain embodiments of the medical adhesive composition disclosed herein comprise star-shaped polyethylene glycol having 2-arms, 4-arms, and/or 8-arms.

In certain embodiments, compositions of the present disclosure comprise a synthetic polymer, and a matrix protein. In preferred embodiments, compositions of the present disclosure comprise a phenol-enriched synthetic polymer and a phenol-modified matrix protein. In certain preferred embodiments, the present disclosure provides compositions comprising phenol-enriched polyethylene glycol and phenol-modified gelatin. In accordance with some forms, 20-80% of the total phenol of the composition is provided by phenol-enriched synthetic polymer. In accordance with certain embodiments, the ratio of phenol groups provided by the phenol-enriched synthetic polymer to phenol groups provided by the phenol-modified matrix protein is about 2:1 to about 1:4, preferably about 1:2. A matrix protein, for example phenol-modified gelatin, may comprise 10% to 50% by weight of the medical adhesive composition, preferably 20% to 40% by weight, more preferably about 30% by weight. A synthetic polymer, for example a phenol-enriched synthetic polymer, may comprise 1% to 20% by weight of the medical adhesive composition, preferably 2% to 10%. In some forms, phenol-enriched 4-arm polyethylene glycol comprises about 5.7% by weight of the medical adhesive composition. In some forms, phenol-enriched 8-arm polyethylene glycol comprises about 3.4% by weight of the medical adhesive composition.

While not wishing to be bound by theory, it is believed that the mechanism involves irradiation of the catalyst to induce an excited state, followed by transfer of an electron from the metal to an electron acceptor. The oxidized metal then extracts an electron from a side chain such as a tyrosine side chain or other phenol group in the matrix protein and/or synthetic polymer to produce, a tyrosyl radical that reacts immediately with a nearby tyrosine to form a dityrosine bond. A direct cross-link (without any bridging moiety) is created quickly in this photo-initiated chemical reaction, without the need for introduction of a primer layer and without the generation of potentially detrimental species such as singlet oxygen, superoxide and hydroxyl radicals. The term “photoactivatable metal-ligand complex” as used herein means a metal-ligand complex in which the metal can enter an excited state when irradiated such that it can donate an electron to an electron acceptor in order to move to a higher oxidation state and thereafter extract an electron from a side chain of an amino acid residue of a matrix protein to produce a free radical without reliance upon the formation of singlet oxygen. Suitable metals include but are not limited to Ru(II), Pd(II), Cu(II), Ni(II)-Mn(II) and Fe(III) in the form of a complex which can absorb light in the visible region, for example, an Ru(II) bipyridyl complex, a Pd(II) porphyrin complex, a sulfonatophenyl Mn(II) complex or a Fe(III) protoporphyrin complex, more particularly, an Ru(II) bispyridyl complex or a Pd(II) porphyrin, in particular, an Ru(II) (bpy)₃ complex such as [Ru(II) (bpy)₃]Cl₂. Efficient cross-linking occurs in the presence of an electron acceptor, and requires only moderate intensity visible light. It has been discovered that a cross-linking reaction may occur in the absence of a photoactivatable metal-ligand complex. Such formulations require extended curing time, for example at least two hours, and potentially up to about 24 hours. In this way, compositions of the present disclosure, with or without a metal ligand complex, may form crosslinks in the absence of light. Thus, the methods disclosed herein may be practiced without irradiating the injected composition with light, such methods require a curing time of at least two hours, and may not be fully crosslinked for about 24 hours.

As used herein the term “electron acceptor” refers to a chemical entity that accepts electron transferred to it and so refers to an easily reduced molecule (or oxidizing agent) with a redox potential sufficiently positive to facilitate the cross-linking reaction. A range of electron acceptors will be suitable. In an embodiment, the electron acceptor is a peracid, a cobalt complex, a cerium (IV) complex, or an organic acid. An exemplary reaction is shown below:

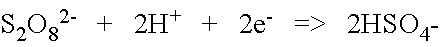

Typically, the electron acceptor is a persulfate, periodate, perbromate or perchlorate compound, vitamin B12, Co(Ill) (NH₃)₈Cl²⁺, cerium (IV) sulphate dehydrate, ammonium cerium (IV) nitrate, oxalic acid or EDTA. Preferably, the persulfate anion is used as the electron acceptor. The standard oxidation-reduction potential for the reaction is 2.1 V, as compared to 1.8 V for hydrogen peroxide (H₂O₂). This potential is higher than the redox potential for the permanganate anion (MnO₄—) at 1.7 V, but slightly lower than that of ozone at 2.2V.

The term “phenol enriched” as applied to a matrix protein or synthetic polymer material herein means that the material has been chemically modified to increase the number of phenolic groups in the material. Thus, “phenol enriched collagen” refers to collagen that has been chemically modified to increase the number of phenolic groups (e.g. tyrosine groups) in the collagen, “phenol enriched gelatin” refers to gelatin that has been chemically modified to increase the number of phenolic groups in the gelatin, “phenol enriched collagen peptide composition” refers to a collagen peptide composition that has been chemically modified to increase the number of phenolic groups in the collagen peptide composition, and “phenol enriched synthetic polymer” refers to synthetic polymer that has been chemically modified to increase the number of phenolic groups in the synthetic polymer. In some aspects, the phenolic groups are tyrosine groups, which can be added for example using a known Bolton Hunter reagent. In some aspects, the phenol enriched material (e.g. synthetic polymer, collagen, gelatin, or collagen peptide composition) will have a P/G value of at least about 7, and in certain forms in the range of about 7 to about 30, or in the range of about 15 to about 30, or in the range of about 18 to about 25, where the P/G value is the number of moles of phenol groups per mole of polymer (synthetic polymer or matrix protein) in the material. The P/G value for a material can be determined using standard techniques, including for example using an absorbance assay at a wavelength of 280 nm. Moderate P/G ranges for the phenol-enriched materials, as recited above, are preferred in some aspects, as modification to higher P/G values has been found to decrease the solubility of the material in aqueous media (see e.g. Example 9 below for phenol enriched gelatin).

In preferred forms, a multi-component system is provided for preparing a photocurable adhesive as described above. A first component can include a liquid carrier, the phenol-enriched synthetic polymer and if present any other polymer(s) containing phenolic groups, and the photoactivatable catalyst; and, a second component can include the electron acceptor. The second component can be in the form a dry powder or in the form of a flowable liquid, for example a flowable liquid including an aqueous medium and the electron acceptor. The first and second components can be mixed to form a flowable photocurable liquid adhesive that, when exposed to visible light, cures by the formation of covalent diphenolic crosslinks between molecules of the polymer.

Certain embodiments herein provide a kit for preparing a photocurable adhesive. The kit can include a first container defining a first chamber within a sterile barrier and containing a sterile liquid preparation in the first chamber. The sterile liquid preparation includes an aqueous liquid such as water or phosphate buffered saline, the phenol-enriched synthetic polymer and if present any other phenolic polymer(s) dissolved in the aqueous liquid, and a photoactivatable catalyst. The kit can further include a second container defining a second chamber within a sterile barrier and containing an electron acceptor in the second chamber. The sterile liquid preparation and the electron acceptor are mixable to prepare a photocurable liquid adhesive effective to form a diphenolic crosslinked polymer hydrogel when photocured. In some forms, the kit can also include a cannulated connector for fluidly connecting the first chamber and the second chamber and/or a visible light source (e.g. a battery-powered light emitting diode visible light source) for curing the photocurable adhesive.

In some forms, the present disclosure provides a medical implant graft comprising a substrate material and a photocurable liquid adhesive as disclosed herein carried by the substrate material, or comprising a substrate material and a cured hydrogel material prepared or preparable by photocuring a photocurable liquid adhesive as disclosed herein. For instance, the photocurable liquid adhesive or the cured hydrogel material can be coated on and/or incorporated within the substrate material. In certain embodiments, such substrate materials can be in the form of a medical wrap or overlay. In certain embodiments, the substrate material comprises a remodelable material. Particular advantage can be provided by including a remodelable collagenous material in or as the substrate material. Such remodelable collagenous materials can be provided, for example, by collagenous materials isolated from a suitable tissue source from a warm-blooded vertebrate, and especially a mammal. Reconstituted or naturally derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth. In some forms, the substrate material comprises a surgical mesh. The substrate may comprise a synthetic material. Suitable synthetic materials include non-bioresorbable or bioresorbable synthetic polymer materials such as polytetrafluroethylene (PTFE, e.g. GORE-TEX material), nylon, polypropylene, polyurethane, silicone, DACRON polymer, polyglycolic acid (PGA), polylactic acid (PLA), polycaprolactone, or others. In some forms, the substrate material may include a collagenous extracellular matrix material and a synthetic material. For example, a synthetic polymer material may be used to stitch layers of collagenous extracellular matrix materials together, or to reinforce one or more layers of collagenous extracellular matrix material. In certain embodiments, a synthetic mesh may be present alongside, or between layers of collagenous extracellular matrix materials.

Suitable bioremodelable materials can be provided by collagenous extracellular matrix materials (ECMs) possessing biotropic properties, including in certain forms angiogenic collagenous extracellular matrix materials. For example, ECMs include materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, materials that include intestinal submucosa, including small intestinal submucosa, stomach submucosa, urinary bladder submucosa, and uterine submucosa. These identified submucosa or other layers can occur in the ECM material alone, or in combination with other materials such as those derived from one or more adjacent layers in the source tissue.

The submucosa-containing ECM can be derived from any suitable organ or other biological structure, including for example submucosa derived from the alimentary, respiratory, intestinal, urinary or genital tracts of warm-blooded vertebrates. Submucosa-containing materials useful in the present invention can be obtained by harvesting such tissue sources and delaminating the submucosa (alone or combined with other materials) from smooth muscle layers, mucosal layers, and/or other layers occurring in the tissue source. For additional information as to submucosal materials useful in the present invention, and its isolation and treatment, reference can be made, for example, to U.S. Pat. Nos. 4,902,508, 5,554,389, 5,993,844, 6,206,931, and 6,099,567.

When a submucosal or other ECM material having differing characteristic sides is used in combination with a coating, the coating can be oriented upon the medical graft on a specified side. For example, in the case of small intestinal submucosa, the coating may be oriented in any manner as described herein, on either the luminal or abluminal side of the small intestinal submucosa.

As prepared, the submucosal material and any other ECM used may optionally retain growth factors or other bioactive components native to the source tissue. For example, the submucosal or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

S Further, in addition or as an alternative to the inclusion of native bioactive components, non-native bioactive components such as those synthetically produced by recombinant technology or other methods, may be incorporated into the submucosal or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient. Alternatively, or additionally, a non-native bioactive component can be included in the coating material of the medical product. When included in the coating, the non-native bioactive component can be added at any point during preparation of the medical product, including being mixed with one or all of the coating components prior to application of the coating to a surface of a layer of a medical material or, alternatively, after the coating is formed, applied, or cross-linked.

A non-native bioactive component can be applied to a submucosal or other ECM tissue by any suitable means. Suitable means include, for example, spraying, impregnating, dipping, etc. The non-native bioactive component can be applied to the ECM tissue either before or after the coating is applied to the material, or both. Similarly, if other chemical or biological components are included in the ECM tissue, the non-native bioactive component can be applied either before, in conjunction with, or after these other components.

Submucosal or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosal or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosal tissue used in the present invention.

In some embodiments herein, the medical implant graft can be a multilaminate medical graft that carries a photocurable liquid adhesive as described herein or a cured hydrogel material prepared or preparable by photocuring a photocurable liquid adhesive as described herein. For example, a plurality of (i.e. two or more) layers of a biocompatible material, for example submucosa-containing or other ECM material, can be bonded together to form a multilaminate structure. Illustratively, two, three, four, five, six, seven, or eight or more layers of a biocompatible material can be bonded together to provide a multilaminate bolster material. The layers of biocompatible material can be bonded together in any suitable fashion, including dehydrothermal bonding under heated, non-heated or lyophilization conditions, stitching, using a photocurable adhesive as described herein, glues or other bonding agents, crosslinking with chemical agents or radiation (including UV radiation), or any combination of these with each other or other suitable methods.

In accordance with some forms, the medical compositions described herein may include one or more additives that alter the performance of the composition. Suitable additives for use herein may be included to improve lubricity, improve the aesthetics of the cross-linked material, reduce inflammation, and/or other beneficial changes. It is within the scope of the present disclosure to provide an adhesive composition having one or more additives, which may provide similar or different advantages. Thus, in certain embodiments, the present disclosure provides medical adhesive compositions including additives for increasing the lubricity of the crosslinked material, such additives include but are not limited to: hyaluronic acid, sodium hyaluronate, and/or chondroitin sulfate. In some forms one or more hydrophilic additives may be included, suitable hydrophilic additives include sugars, for example fructose. Hydrophilic additives may cause the medical adhesive to form a more robust layer upon crosslinking. In some forms, one or more additives may be included, which contribute additional resistance to adhesion of the crosslinked adhesive to surrounding patient tissues, for example zwitterionic polymers.

The present disclosure provides methods of making an implantable medical graft. In some forms, the present disclosure provides methods including the step of applying a medical composition to a substrate as described herein. Such applying can be achieved in any suitable fashion, for example spraying, brushing, soaking, rolling, injecting, or any other suitable technique. In use, a medical graft may be applied to patient tissue with an adhesive composition applied on the exterior and/or interior (e.g. toward patient tissue) of the medical graft. After the medical composition is applied to the substrate, the resulting construct may then be irradiated to form a cross-linked construct. In accordance with some forms, methods of the present disclosure may include the step of irradiating a substrate as described herein. The present disclosure provides coating materials that form cross-links under moderate intensity visible light. In certain embodiments, irradiation may be performed using white light, for example 450 nm nominal wavelength light.

The implantable medical grafts described herein have broad application. In some aspects, inventive products will find use as precursor materials for the later formation of a variety of other medical products, or components thereof. Medical gratis and materials that are already commercially available can be modified in accordance with the present invention as well. In certain embodiments, inventive products are useful in procedures to replace, augment, support, repair, and/or otherwise suitably treat diseased or otherwise damaged or defective patient tissue. Some of the illustrative implantable medical grafts described herein will be useful, for example, in treating diseased or damaged nerve tissue and grafts as disclosed herein can be developed and used in many other medical contexts. In this regard, when used as a medical graft, the devices disclosed herein can be utilized in any procedure where the application of the graft to a bodily structure provides benefit to the patient. Illustratively, graft materials of the invention can be processed into various shapes and configurations, for example, into a variety of differently shaped urethral slings, surgical bolster or reinforcement materials (e.g., for use in tissue resection and similar procedures), wound products and other grafts and graft-like materials.

In certain embodiments, the medical adhesive is present in a uniform layer covering one or more surfaces of the underlying substrate. In some forms, the substrate is generally sheet-form having a first surface and a second surface. In certain embodiments, the medical adhesive is present on the first surface while the second surface is free of medical adhesive. In some forms, the medical adhesive is present on both the first a second surfaces. In accordance with certain embodiments, the medical adhesive is soaked into the substrate, such that the medical adhesive permeates the matrix structure of the substrate prior to crosslinking. The medical adhesive can be present in a variety of forms, for example in certain embodiments the medical adhesive is patterned on the surface of the substrate. The medical adhesive may be present in any suitable pattern, for example lines, cross-hatching, dots, or dots. Thus, in some forms a surface may have one or more coated portions and one or more uncoated portions. Such patterns may be advantageous, for example, to allow portions of the substrate to contact patient tissue, or to promote tissue sealing to only a portion of the substrate.

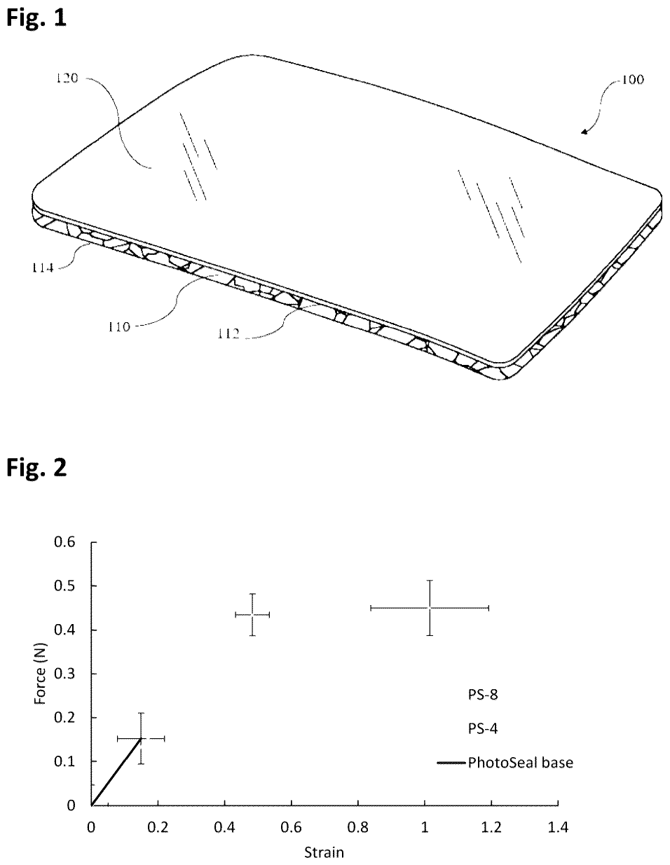

With reference now to the embodiment depicted in FIG. 1, shown is one embodiment of an implantable medical graft 100. In the illustrated embodiment, the medical graft comprises a substrate 110 and an adhesive material 120. In the illustrated embodiment, the adhesive material is coated in a substantially uniform layer disposed on a first face 112 of the substrate. A second face 114 of the substrate is free of the adhesive material. As shown, the adhesive material is layered on at least one surface of the substrate, however as described herein it is within the scope of the disclosure to provide a substrate material and an adhesive in any suitable form, e.g. partially coated, fully coated, saturated, etc. In the illustrated embodiment, the substrate and the coating are present in substantially sheet form.

In some forms, the medical composition as described herein may be present in a uniform layer over substantially all of a coated a face of a substrate material. In other embodiments, the medical composition is patterned unto the substrate face such that the face of the substrate material has coated portions and uncoated portions. The medical composition may be applied in any suitable pattern, for example, linear segments extending from one end of the graft to the other. In some forms, the medical composition is present in shaped sections, such as one or more circular or polygonal shaped coated portions on the surface of the substrate.

It is also within the scope of the present disclosure to provide a medical graft material comprising a medical composition on both faces of a sheet-form substrate material. For example, a substrate material may be provided having a first face opposing a second face, and a first medical composition layer is deposited on the first face and a second medical composition layer is deposited on the second face. As disclosed above the medical composition layers may coat the entire face or only a coated portion leaving uncoated portions. The two faces may be coated in the same fashion. e.g. each having a uniform or patterned coating. Alternatively, the two faces may be coated differently, for example, a first surface may have a uniform coating while the second face has a patterned coating.

In preferred forms, a multi-component system is provided for preparing a photocurable adhesive as described above. A first component can include water, the polymer(s) containing phenolic groups and the metal ligand complex; and, a second component can include the electron acceptor. A second component can be in the form a dry powder or in the form of a flowable liquid, for example a flowable liquid including an aqueous medium and the electron acceptor. The first and second components can be mixed to form a flowable photocurable adhesive that, when exposed to visible light, cures by the formation of covalent diphenolic crosslinks between molecules of the polymer.

Certain embodiments herein provide a kit for preparing a photocurable adhesive. The kit can include a first container defining a first chamber within a sterile barrier and containing a sterile liquid preparation in the first chamber. The sterile liquid preparation includes an aqueous liquid, the phenolic polymer(s) dissolved in the aqueous liquid such as water or phosphate buffered saline, and a metal ligand complex. The kit can further include a second container defining a second chamber within a sterile barrier and containing an electron acceptor in the second chamber. The sterile liquid preparation and the electron acceptor are mixable to prepare a photocurable liquid adhesive effective to form a diphenolic crosslinked polymer hydrogel when photocured. In some forms, the kit can also include a cannulated connector for fluidly connecting the first chamber and the second chamber and/or a visible light source (e.g. a battery-powered light emitting diode visible light source) for curing the photocurable adhesive.

In some forms, the sterile liquid preparation in the first chamber includes synthetic polymer, a phenol enriched synthetic polymer, collagen, phenol enriched collagen, gelatin, phenol enriched gelatin, a collagen peptide composition, or a phenol enriched collagen peptide composition. These polymer materials can be used either singly or in combination. For example, the photocurable adhesive may include a combination of synthetic polymer, a phenol enriched synthetic polymer, or a combination of collagen and phenol enriched collagen, a combination of gelatin and phenol enriched gelatin, or a combination of a collagen peptide composition and a phenol enriched collagen peptide composition. In each case, the dry weight ratio of the parent polymeric material and its phenol enriched counterpart can be in the range of about 1:10 to about 10:1, or about 1:5 to about 5:1, or in some forms about 1:5 to about 1:2. Mixtures of two or more of collagen, gelatin, and a collagen peptide composition (each in its native form without phenol enrichment or as a phenol enriched polymeric material) can also be used.

In addition, or alternatively, the sterile liquid preparation that includes collagen, phenol enriched collagen, gelatin, phenol enriched gelatin, a collagen peptide composition, or a phenol enriched collagen peptide composition, or any mixture of two or more thereof, can exhibit the property of not gelling at 20° C., for example exhibiting no thermoreversible gelation activity upon cooling, or having a thermoreversible gelation temperature below 20° C., or below 15° C. In some forms, the sterile liquid preparation comprises gelatin, phenol enriched gelatin, or a mixture thereof, and the liquid preparation also includes an agent that inhibits the thermoreversible gelling of the gelatin (when present) and of the phenol enriched gelatin (when present). Urea is a preferred agent that inhibits this thermoreversible gelling, and can be used for example at a concentration in the range of about 1 molar to 5 molar in the liquid preparation, more typically about 3 molar to about 4.5 molar, and in some forms about 3.8 molar to about 4.5 molar. In other forms, the sterile liquid preparation includes a collagen peptide composition and/or a phenol enriched collagen peptide composition, that has an average molecular weight (Mw) below about 20,000 kilodaltons, more preferably below about 15,000 kilodaltons, and typically in the range of about 2,000 to about 12,000 kilodaltons. In these forms, the collagen peptide composition can exhibit no thermoreversible gelation activity upon cooling to 20° C. (or in some typical forms at any temperature), allowing the liquid preparation to remain a liquid at a temperature of 20° C., or at a temperature of 15° C. It will be understood that the liquid preparation may also remain a liquid at temperatures below these specified temperatures, and in general may remain a liquid throughout a temperature range expected to encompass room temperature storage and normal use temperatures, for example in the range of about 20° C. to about 37° C.

The sterile liquid preparation can include the polymer(s) containing phenolic groups in any suitable concentration. In some forms, the total concentration of the polymer(s) present in the sterile liquid preparation will be in the range of about 1% to about 40% weight/volume, more typically about 10% to about 40% weight:volume. In certain preferred forms, the sterile liquid preparation will include collagen, phenol enriched collagen, gelatin, phenol enriched gelatin, a collagen peptide composition, a phenol enriched collagen peptide composition, or any combination thereof, at a concentration in the range of about 20% to about 35% weight/volume, or in the range of about 25% to about 35% weight volume. In such forms, the sterile liquid preparation, and photocurable liquid adhesives prepared using it, can be a flowable viscous liquid, for example having a viscosity at 20° C. of greater than about 300 centipoise, or greater than about 500 centipoise, and typically in the range of about 500 to about 20000 centipoise or in the range of about 1000 to about 10000 centipoise.

The sterile liquid preparation can include the metal ligand complex in a suitable amount to catalyze the formation of covalent crosslinks in the formation of the covalently crosslinked hydrogel by photocuring. Where a Ru(II) (bpy)₃ complex such as [Ru(II) (bpy)₃]Cl2 is used as the metal ligand complex, preferred sterile liquid preparations will include it at a concentration in the range of about 0.2 to about 2 mM, more desirably about 0.4 to about 1 mM. Where the electron acceptor to be mixed with the sterile liquid preparation is in dry powder form, the prepared photocurable liquid adhesive will have these same concentrations of the metal ligand complex. Where the electron acceptor is provided in a solution to be combined with the sterile liquid preparation, the concentration of the metal ligand complex in the prepared photocurable liquid adhesive will be reduced relative to that in the sterile liquid preparation. In some such forms, the volume of the sterile liquid preparation, the volume of the solution of electron acceptor, and the concentration of the metal ligand complex in the sterile liquid preparation, can be selected to provide a concentration of the metal ligand complex in the prepared photocurable liquid adhesive that is within the above-referenced concentration range values given for the sterile liquid preparation.

The sterile liquid preparation can have been terminally sterilized within the first chamber to render the liquid preparation sterile (e.g. using sterilizing radiation applied to a package containing the first container), but in some preferred forms the liquid preparation is sterilely prepared, for example including passage of the liquid preparation through a sterile filter, and then filled into the first chamber in a sterile filling operation. Such sterilely-filled liquid preparations in the first chamber can therefore be free from exposure to sterilizing radiation, and thus can be free from any degradation of the polymer(s) containing phenol groups caused by the sterilizing radiation. In some forms, the liquid preparation can be in a heated condition to reduce its viscosity during passage through the sterile filter. Also, in some forms, the first container having the first chamber containing the sterilely-filled liquid preparation can be sealed within a sterile barrier package under sterile conditions. Further, such sterile barrier package is preferably impermeable to visible light, as can be provided for example by a foil pouch package.

To promote a further understanding of embodiments disclosed herein and their features and advantages, the following specific Examples are provided. It will be understood that these examples are illustrative and not limiting in nature. In the following examples, amine-terminated star-shaped (2-arm, 4-arm, or 8-arm of various molecular mass) polyethylene glycol (PEG) was chemically modified so each arm terminated in a phenol group. Phenol-modified gelatin and phenol-modified PEG were mixed in aqueous solution and combined with an oxidizer (sodium persulfate) and a photo-catalyst (ruthenium). In general, incorporating PEG created a material that was softer (lower Young's modulus), more stretchy (higher strain at failure), and stronger (higher stress at failure).

### Example 1

### Tensile Testing of Cured Adhesive

Various compositions as detailed below were molded and photo-cured into a dog bone shape, and tested under tension (ASTM D412). “Photoseal base” is a phenol-modified gelatin formulation without PEG (˜30 w %). “PS-4” is phenol-modified gelatin combined with phenol-modified 4-arm PEG. “PS-8” is phenol-modified gelatin combined with phenol-modified 8-arm PEG. Both PEG types had a molecular mass of 10,000 Da before modification. The ratio of PEG phenol groups to gelatin phenol groups was 1:2 (5.7 w % 4-arm, 3.4 w % 8-arm). Samples were tested under tension until failure. The results are shown in FIG. 2, indicating the force and strain at failure, and the slope of the line indicates the stiffness (Young's Modulus). n=7. It was discovered that incorporating modified PEG reduced the stiffness of the material and significantly increased the stress and strain at failure.

### Example 2

### Young's Modulus of Cured Material Across PEG Shape and Molecular Mass

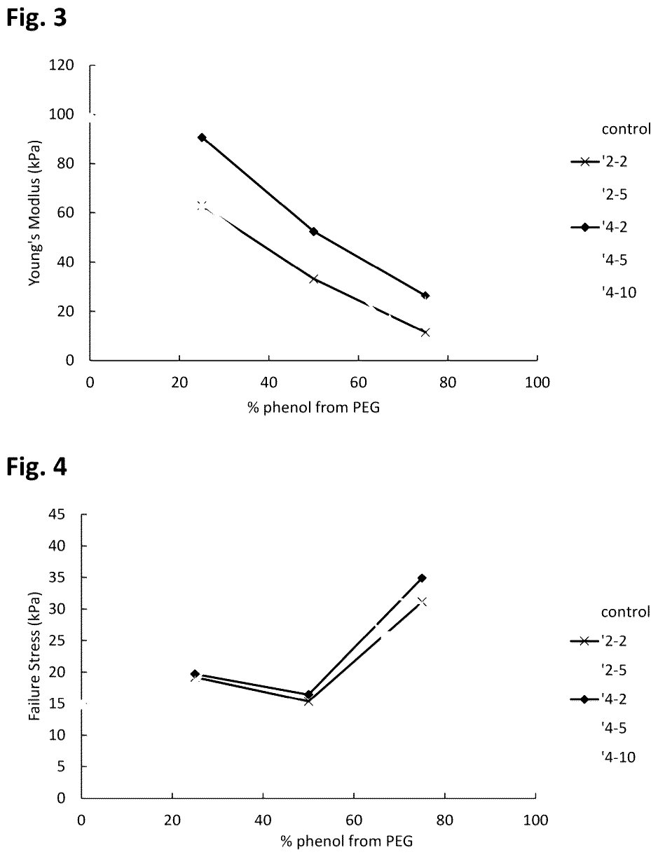

2-armed or 4-arms PEG with a molecular mass of 2,000 Da, 5,000 Da, or 10,000 Da was chemically modified so that each arm terminated in a phenol group. As shown in FIG. 3, various formulations were analyzed. In the legend, X-Y indicated X-armed PEG at Y kDa molecular mass. PEG was incorporated into a standard gelatin formulation (100% phenol-modified gelatin, ˜30 w %) at various concentrations so that the percent of total phenol in the mixture that came from the PEF groups varied between 20 and 80% (a higher % of phenol from PEG indicated a higher concentration of PEG). Gelatin and ruthenium concentration were kept constant. Sodium persulfate concentration in the mixture was 0.1M for the control (no PEG) and 0.2M for the PEG formulations. The control group is shown as a line for ease of interpretation. n=1. It was discovered that all groups containing PEG were softer than the control group. As PEG concentration increased, stiffness decreased. Groups with 4-arms PEG were stiffer than groups with 2-armed PEG.

### Example 3

### Stress at Failure of Cured Material Across PEG Shape and Molecular Mass

2-armed or 4-arms PEG with a molecular mass of 2,000 Da, 5,000 Da, or 10,000 Da was chemically modified so that each arm terminated in a phenol group. As shown in FIG. 4, various formulations were analyzed. In the legend, X-Y indicated X-armed PEG at Y kDa molecular mass. PEG was incorporated into a standard gelatin formulation (100% phenol-modified gelatin, ˜30 w %) at various concentrations so that the percent of total phenol in the mixture that came from the PEF groups varied between 20 and 80% (a higher % of phenol from PEG indicated a higher concentration of PEG). Gelatin and ruthenium concentration were kept constant. Sodium persulfate concentration in the mixture was 0.1M for the control (no PEG) and 0.2M for the PEG formulations. The control group is shown as a line for ease of interpretation, n=1. It was discovered that all groups containing PEG had a higher stress at failure than the control, 2,000 Da molecular mass PEG groups had increased stress at failure with higher concentration PEG, 5,000 Da and 10,000 Da molecular mass PEG groups had maximum stress at failure at an intermediate concentration PEG.

### Example 4

### Stress at Failure of Cured Material Across PEG Shape and Molecular Mass

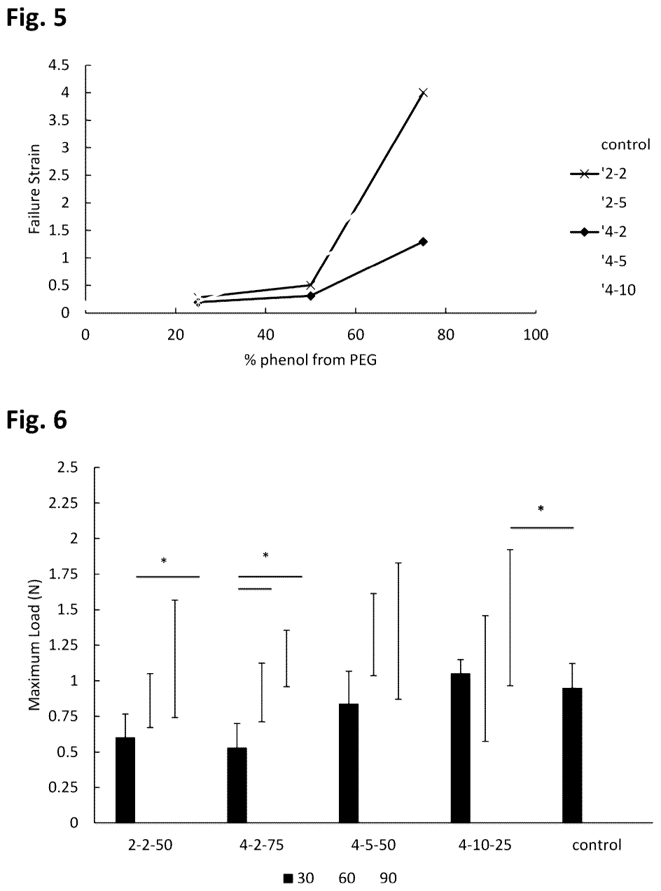

2-armed or 4-arms PEG with a molecular mass of 2,000 Da, 5,000 Da, or 10,000 Da was chemically modified so that each arm terminated in a phenol group. As shown in FIG. 5, various formulations were analyzed. In the legend, X-Y indicated X-armed PEG at Y kDa molecular mass. PEG was incorporated into a standard gelatin formulation (100% phenol-modified gelatin, ˜30 w %) at various concentrations so that the percent of total phenol in the mixture that came from the PEF groups varied between 20 and 80% (a higher % of phenol from PEG indicated a higher concentration of PEG). Gelatin and ruthenium concentration were kept constant. Sodium persulfate concentration in the mixture was 0.1M for the control (no PEG) and 0.2M for the PEG formulations. The control group is shown as a line for ease of interpretation. n=1. It was discovered that all groups containing PEG had a higher strain at failure than the control. All groups had increased strain at failure with increased PEG concentration.

### Example 5

### Maximum Force of End-to-End Wound Closure

Various formulations of adhesive compositions as detailed below were applied to vacuum pressed hydrated small intestine submucosa (SIS) patched to form an end-to-end closure and tested for maximum force (ASTM F2458) at failure. The formulations were cured for 30 seconds, 60 seconds, or 90 seconds. With reference to the results detailed in FIG. 6, X-Y-Z in the horizontal axis indicates X armed PEG, Y kDa molecular mass PEG, and Z % phenol from PEG. PEG was incorporated into a standard gelatin formulation (100% phenol-modified gelatin, ˜30 w %). Gelatin and ruthenium concentration were kept constant. Sodium persulfate concentration in the mixture was 0.1M for the control (no PEG) and 0.2M for the PEG formulations. n=6, *indicates p<0.05. It was discovered that increased cure time increased the maximum force of the closure.

### Example 6

### End-to-End Closure with SIS Overlay

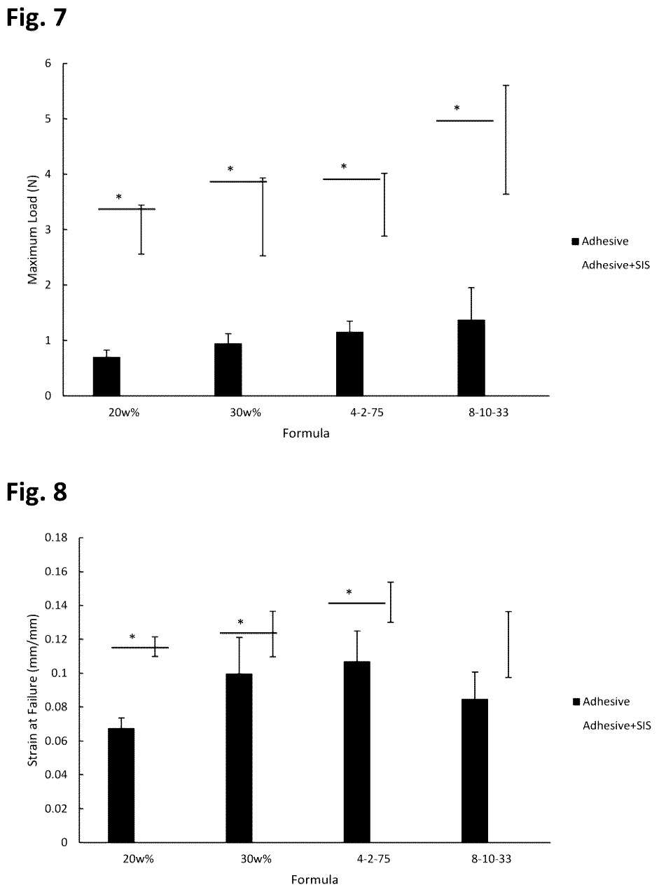

Wound closure analogs were prepared as detailed in Example 5 above or with a square of SIS overlaid on top of the closure prior to curing (“Adhesive+SIS”). Maximum load and strain at failure was determined for tested closures (ASTM F2458). With reference to the results shown in FIGS. 7 and 8, 20 w-% and 30 w % groups are adhesive without PEG, with 20 w % and 30 w % gelatin, respectively (100% phenol-modified gelatin). X-Y-Z in the horizontal axis indicates X armed PEG, Y kDa molecular mass PEG, and Z % phenol from PEG. PEG was incorporated into a standard gelatin formulation (100% phenol-modified gelatin, ˜30 w %). Gelatin and ruthenium concentration were kept constant. Sodium persulfate concentration in the mixture was 0.1M for the control (no PEG) and 0.2M for the PEG formulations. Adhesive was cured for 90 seconds. n=4, *indicates p<0.05. It was discovered that use of an SIS overlay significantly increases the strength of the wound closure in all groups.

### Example 7

### Maximum Force of Wound Closure of Porcine Optic Nerve

Nerves were closed with adhesive alone (“direct”), with an SIS wrap and adhesive applied on the exterior of the wrap (“wrap outside”) or on the interior of the wrap (“wrap inside”). “Control” is a 30 w % gelatin formulation (100% phenol-modified gelatin). X-Y-Z in the horizontal axis indicates X armed PEG, Y kDa molecular mass PEG, and Z % phenol from PEG. PEG was incorporated into a standard gelatin formulation (100% phenol-modified gelatin, ˜30 w %). Gelatin and ruthenium concentration were kept constant. Sodium persulfate concentration in the mixture was 0.1M for the control (no PEG) and 0.2M for the PEG formulations. Adhesive was cured for 90 seconds. n=4.

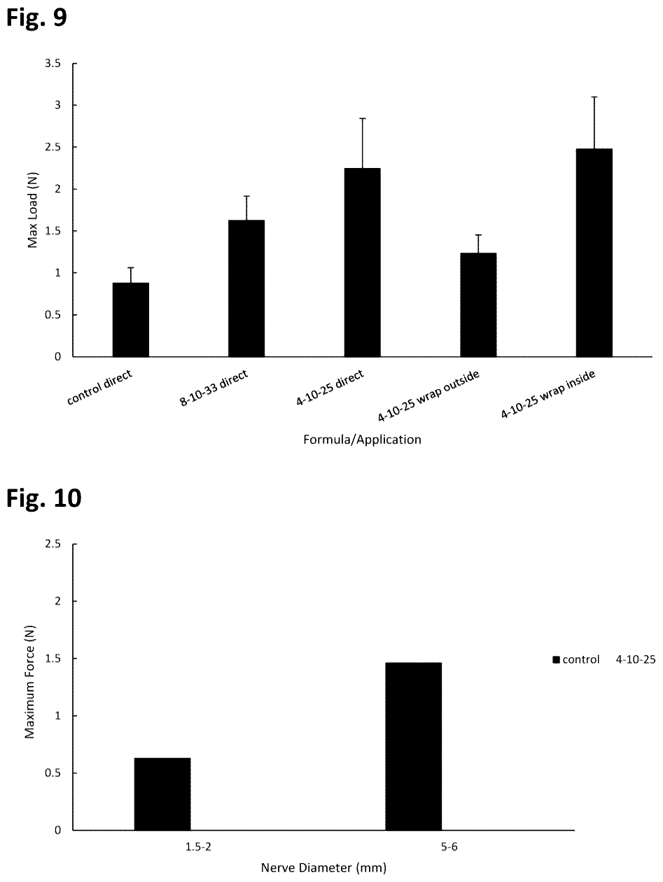

With reference to the results shown in FIG. 9, both PEG groups have a higher repair strength than the group without PEG. Using an STS wrap did not improve repair strength. For comparison, primary nerve repair with four 8-0 sutures has a maximum load of 1.5 N, and repair with a nerve conduit using four 8-0 sutures on each end of the conduit has a maximum load of 2 N.

### Example 8

### Maximum Force of Wound Closure of Human Decellularized Nerve Graft

Human decellularized nerve gratis were closed with a gelatin or PEG formulation. Grafts were closed with direct repair only. With reference to the results shown in FIG. 10, “control” is a 30 w % gelatin formulation (100% phenol-modified gelatin). X-Y-Z in the horizontal axis indicates X armed PEG, Y kDa molecular mass PEG, and Z % phenol from PEG. PEG was incorporated into a standard gelatin formulation (100% phenol-modified gelatin, ˜30 w %). Gelatin and ruthenium concentration were kept constant. Sodium persulfate concentration in the mixture was 0.1M for the control (no PEG) and 0.2M for the PEG formulations. Adhesive was cured for 90 seconds. It was discovered that the group with PEG had a higher repair strength than adhesive without PEG.

### Example 9

### Phenol Enriched Gelatin Prepared with Bolton Hunter Reagent

Nippi MediGelatin (derived from porcine skin; Mw approximately 100 kilodaltons) was dissolved in high purity water with 6.18 g/L boric acid, 9.54 g/L sodium borate, and 4.38 g/L sodium chloride at 30° C. and 60° C. at a concentration of 10 g/L in a IL reaction volume (n=1). 200 mL aliquots of this solution were extracted into 500 mL Erlenmeyer flasks and combined with 3.5 mL of a Bolton Hunter reagent/DMSO solution (Bolton Hunter reagent N-succinimidyl-3-[4-hydroxyphenyl]propionate). The Bolton Hunter/DMSO solution was prepared at a concentration such that the final concentration of Bolton Hunter reagent in the gelatin solution ranged from 0.2 to 5 g/L. The mixture was reacted at 40° C. in a shaken incubator for two hours. The solution was dialyzed, dried, and analyzed for phenol content using absorbance at 280 nm. Unless noted otherwise, groups had a replicate size of n=3. Each group was evaluated for normality and compared across groups for equal variance. Groups were then compared for statistical difference using a one-way ANOVA (α=0.05) and Tukey post-hoc tests.

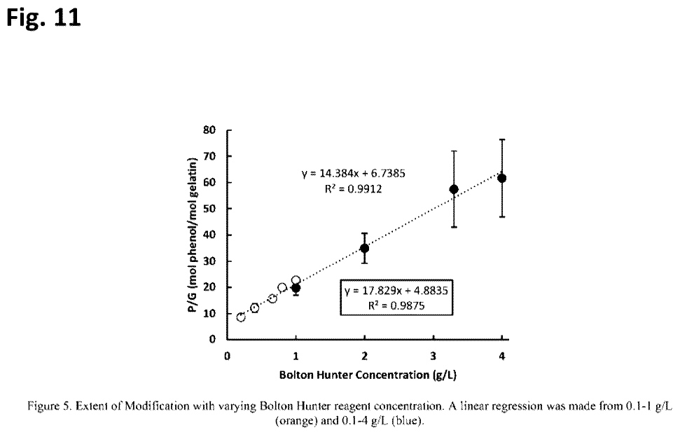

The results are summarized in FIG. 11. P/G values (mole phenol/mole gelatin) for the modified materials ranged from just under 10 to about 60 (the unmodified gelatin had a P/G value of about 3). At all concentrations at and below 1 g/L Bolton Hunter, the resulting phenol enriched gelatin was soluble in PBS. The relationship between Bolton Hunter concentration and extent of phenol enrichment was linear, with a 10% change in Bolton Hunter concentration causing a ˜6% change in the molar ratio of phenol to gelatin. At Bolton Hunter concentrations at 2 g/L and higher, the relationship became less linear and the measurements had higher standard deviation. In addition, the modified gelatin became less soluble in PBS, with protein modified at 5 g/L becoming completely insoluble. These results indicate that a severe excess of Bolton Hunter reagent and a resulting very high P/G value for the phenol enriched protein can decrease the solubility of the modified protein in an aqueous medium.

### Example 10

### Phenol Enriched Gelatin Prepared by Carbodiimide Reaction

Gelatin was modified to include additional phenol groups using EDC (1-ethyl-3-(3-dimethylaminopropyl)carbodiimide hydrochloride), NHS (N-hydroxysuccinimide), and HPPA (3-(4-Hydroxyphenyl)propionic acid). A precipitate was first prepared using a 5:2:1 ratio of NHS:HDC:HPPA at concentrations of 325 mM NUIS, 130 mM EDC and 65 mM HPPA. First, HPPA was solubilized in a 0.1M MES, 0.9% Sodium Chloride. pH 4.7 buffer on a stir plate at 200 rpm. Once the HPPA was dissolved, the EDC and NHS were added to the solution. After 15-20 minutes a precipitate began to form. The solution was allowed to react for 2 to 4 hours and then vacuum filtered. Following double filtration of the solution, the precipitate captured on the filter paper was allowed to dry in a fume hood for at least 24 hours. Once the precipitate was dry, it was utilized as solubilized in DMSO in 4× mass in place of the Bolton-Hunter Reagent to add phenolic groups to the gelatin.

Modified gelatin prepared using the precipitate in place of Bolton-Hunter reagent was formulated into photocurable adhesive compositions using a phosphate buffered saline medium, bipyridyl) ruthenium (II) chloride hexahydrate and sodium persulfate. Photocurable adhesive formulations of having 5:1, 1:1 and 0:1 ratios of unmodified gelatin to modified gelatin were prepared. The thus prepared photocurable adhesive demonstrated the ability to cure under visible light.

### Embodiments

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.
1. A medical composition comprising:
   a photoactivatable catalyst,
   an electron acceptor, and
   a phenol-enriched synthetic polymer; and
   a liquid carrier.
2. The medical composition of embodiment 1, wherein said synthetic polymer comprises polyethylene glycol, polyvinyl alcohol, polylactic acid, polyglycolic acid, poly(lactic-co-glycolic acid), and/or poly(glycerol sebacate).
3. The medical composition of any one of the preceding embodiments wherein said synthetic polymer comprise polyethylene glycol and/or wherein said synthetic polymer is a multi-arm synthetic polymer which has been chemically modified so that each arm terminates in a phenol group.
4. The medical composition of embodiment 3, wherein said polyethylene glycol comprises a multi-arm polyethylene glycol which has been chemically modified so that each arm terminates in a phenol group.
5. The medical composition of embodiment 4, wherein said multi-arm polyethylene glycol comprises a 2-arm polyethylene glycol.
6. The medical composition of embodiment 4, wherein said multi-arm polyethylene glycol comprises a 4-arm polyethylene glycol.
7. The medical composition of embodiment 4, wherein said multi-arm polyethylene glycol comprises an 8-arm polyethylene glycol.
8. The medical composition of any one of the preceding embodiments, wherein said electron acceptor comprises sodium persulfate.
9. The medical composition of any one of embodiments I through 8, wherein said photoactivatable catalyst comprises a photoactivatable metal ligand complex.
10. The medical composition of any one of the preceding embodiments, further comprising gelatin.
11. The medical composition of embodiment 10, wherein said gelatin comprises phenol-modified gelatin.
12. The medical composition of embodiment 11, wherein said phenol-enriched synthetic polymer and said phenol-modified gelatin are present in amounts such that a ratio of phenol groups of said phenol-enriched synthetic polymer and phenol groups of said phenol-modified gelatin is about 1:2.
13. The medical composition of embodiment 11, wherein 20% to 80% of the total phenol in the composition is provided by said phenol-enriched synthetic polymer.
14. The medical composition of any one of the preceding embodiments, wherein said phenol-enriched synthetic polymer has a molecular mass of 2 kDa to 10 kDa.
15. The medical composition of any one of the preceding embodiments, wherein said photoactivatable metal-ligand complex comprises ruthenium tris-bipyridyl chloride.
16. A method of crosslinking a synthetic polymer, the method comprising:
   irradiating a composition comprising a photoactivatable catalyst, an electron acceptor, and a phenol-enriched synthetic polymer to initiate a cross-linking reaction.
17. The method of embodiment 16, wherein said irradiating comprises exposing the composition to visible light for a duration sufficient to initiate a cross-linking reaction.
18. The method of embodiment 17, wherein the duration comprises at least 10 seconds.
19. The method of embodiment 18, wherein the duration comprises 10 to 120 seconds.
20. The method of any one of embodiments 16 through 19, wherein the synthetic polymer comprises polyethylene glycol, polyvinyl alcohol, polylactic acid, polyglycolic acid, poly(lactic-co-glycolic acid), and/or poly(glycerol sebacate).
21. The method of any one of the embodiments 16 through 20 wherein the synthetic polymer comprises polyethylene glycol and/or wherein the synthetic polymer is a multi-arm synthetic polymer which has been chemically modified so that each arm terminates in a phenol group.
22. The method of embodiment 21, wherein the polyethylene glycol comprises a multi-arm polyethylene glycol which has been chemically modified so that each arm terminates in a phenol group.
23. The method of embodiment 22, wherein the multi-arm polyethylene glycol comprises a 2-arm polyethylene glycol.
24. The method of embodiment 22, wherein the multi-arm polyethylene glycol comprises a 4-arm polyethylene glycol.
25. The method of embodiment 22, wherein the multi-arm polyethylene glycol comprises an 8-arm polyethylene glycol.
26. The method of any one of embodiments 16 through 25, wherein the electron acceptor comprises sodium persulfate.
27. The method of any one of embodiments 16 through 26, wherein said photoactivatable catalyst comprises a photoactivatable metal ligand complex.
28. The method of any one of embodiments 16 through 27, wherein the composition further comprising gelatin.
29. The method of embodiments 28, wherein the gelatin comprises phenol-modified gelatin.
30. The method of embodiment 29, wherein the phenol-enriched synthetic polymer and the phenol-modified gelatin are present in amounts such that a ratio of phenol groups of the phenol-enriched synthetic polymer and phenol groups of the phenol-modified gelatin is about 1:2.
31. The method of embodiment 29, wherein 20% to 80% of the total phenol in the composition is provided by the phenol-enriched synthetic polymer.
32. The method of any one of embodiments 16 through 31, wherein the phenol-enriched synthetic polymer has a molecular mass of 2 kDa to 10 kDa.
33. The method of embodiment 27, wherein the photoactivatable metal-ligand complex comprises ruthenium tris-bipyridyl chloride.
34. The method of any one of embodiments 16 through 33, wherein the composition is carried by a substrate suitable for implantation.
35. The method of embodiment 34, wherein the substrate comprises an extracellular matrix material.
36. A medical implant comprising:
   a substrate suitable for implantation;
   a medical adhesive carried by said substrate, said medical adhesive comprising:
      a photoactivatable catalyst,
      an electron acceptor, and
      a phenol-enriched synthetic polymer.
37. The medical implant of embodiment 36, wherein said substrate comprises an extracellular matrix material.
38. The medical implant of embodiment 37, wherein said extracellular matrix material comprises submucosa, renal capsule membrane, dermal collagen, dura mater, pericardium, fascia lata, serosa, peritoneum or basement membrane layers, or including liver basement membrane.
39. The medical implant of any one of embodiments 36 through 38, wherein the extracellular matrix material is in sheet form, and wherein said medical adhesive is carried on a first surface of the sheet form extracellular matrix material.
40. The medical implant of any one of embodiments 36 through 39, wherein the medical adhesive is a crosslinked hydrogel.
41. The medical implant of any one of embodiments 36 through 40, wherein said synthetic polymer comprises polyethylene glycol, polyvinyl alcohol, polylactic acid, polyglycolic acid, poly(lactic-co-glycolic acid), and/or poly(glycerol sebacate).
42. The medical implant of any one of embodiments 36 through 41, wherein said synthetic polymer comprises polyethylene glycol and/or wherein the synthetic polymer is a multi-arm synthetic polymer which has been chemically modified so that each arm terminates in a phenol group.
43. The medical implant of embodiment 42, wherein said polyethylene glycol comprises a multi-arm polyethylene glycol which has been chemically modified so that each arm terminates in a phenol group.
44. The medical implant of embodiment 43, wherein said multi-arm polyethylene glycol comprises a 2-arm polyethylene glycol.
45. The medical implant of embodiment 43, wherein said multi-arm polyethylene glycol comprises a 4-arm polyethylene glycol.
46. The medical implant of embodiment 43, wherein said multi-arm polyethylene glycol comprises an 8-arm polyethylene glycol.
47. The medical implant of any one of embodiments 36 through 46, wherein said electron acceptor comprises sodium persulfate.
48. The medical implant of any one of embodiments 36 through 47, wherein said photoactivatable catalyst comprises a photoactivatable metal ligand complex.
49. The medical implant of any one of embodiments 36 through 48, further comprising gelatin.
50. The medical implant of embodiment 49, wherein said gelatin comprises phenol-modified gelatin.
51. The medical implant of embodiment 50, wherein said phenol-enriched synthetic polymer and said phenol-modified gelatin are present in amounts such that a ratio of phenol groups of said phenol-enriched synthetic polymer and phenol groups of said phenol-modified gelatin is about 1:2.
52. The medical implant of embodiment 50, wherein 20% to 80% of the total phenol in the composition is provided by said phenol-enriched synthetic polymer.
53. The medical implant of any one of embodiments 36 through 52, wherein said phenol-enriched synthetic polymer has a molecular mass of 2 kDa to 10 kDa.
54. The medical implant of embodiment 48, wherein said photoactivatable metal-ligand complex comprises ruthenium tris-bipyridyl chloride.
55. A method of preparing a medical adhesive, the method comprising:
   combining a phenol-enriched synthetic polymer, a phenol-modified gelatin, a photoactivatable catalyst and, an electron acceptor.
56. The method of embodiment 55, wherein the phenol-enriched synthetic polymer comprises polyethylene glycol, polyvinyl alcohol, polylactic acid, polyglycolic acid, poly(lactic-co-glycolic acid), and/or poly(glycerol sebacate).
57. The method of embodiment 56 wherein the phenol-enriched synthetic polymer comprises polyethylene glycol and/or wherein the synthetic polymer is a multi-arm synthetic polymer which has been chemically modified so that each arm terminates in a phenol group.
58. The method of embodiment 57, wherein the polyethylene glycol comprises a multi-arm polyethylene glycol which has been chemically modified so that each arm terminates in a phenol group.
59. The method of embodiment 58, wherein the multi-arm polyethylene glycol comprises a 2-arm polyethylene glycol.
60. The method of embodiment 58, wherein the multi-arm polyethylene glycol comprises a 4-arm polyethylene glycol.
61. The method of embodiment 58, wherein the multi-arm polyethylene glycol comprises an 8-arm polyethylene glycol.
62. The method of any one of embodiments 55 through 61, wherein the electron acceptor comprises sodium persulfate.
63. The method of any one of embodiments 55 through 62, wherein the photoactivatable catalyst comprises a photoactivatable metal ligand complex.
64. The method of any one of embodiments 55 through 63, wherein the phenol-enriched synthetic polymer and the phenol-modified gelatin are present in amounts such that a ratio of phenol groups of the phenol-enriched synthetic polymer and phenol groups of the phenol-modified gelatin is about 1:2.
65. The method of any one of embodiments 55 through 63, wherein 20% to 80% of the total phenol in the composition is provided by the phenol-enriched synthetic polymer.
66. The method of any one of embodiments 55 through 65, wherein the phenol-enriched synthetic polymer has a molecular mass of 2 kDa to 10 kDa.
67. The method of embodiment 63, wherein the photoactivatable metal-ligand complex comprises ruthenium tris-bipyridyl chloride.
68. The method of any one of embodiment 55 through 66, comprising:
   coating the medical adhesive onto a substrate suitable for implantation.
69. The method of embodiment 68, wherein the substrate comprises an extracellular matrix material.
70. A method of joining and/or sealing tissues in a surgical procedure, the method comprising:
   applying a medical composition as described in any one of claims 1 through 15 to a tissue portion; and
   irradiating the tissue medical composition to initiate a cross-linking reaction between one or more endogenous proteins and the phenol-enriched synthetic polymer to seal the tissue portion or join the tissue portion to an adjacent tissue portion.

The use of the terms “a” and “an” and “the” and similar referents in the context of describing the invention especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A medical composition comprising:
a photoactivatable catalyst,
an electron acceptor, and
a phenol-enriched synthetic polymer; and
a liquid carrier.

2. The medical composition of claim 1, wherein said synthetic polymer comprises polyethylene glycol, polyvinyl alcohol, polylactic acid, polyglycolic acid, poly(lactic-co-glycolic acid), and/or poly(glycerol sebacate).

3. The medical composition of claim 2 wherein said synthetic polymer is a multi-arm synthetic polymer which has been chemically modified so that each arm terminates in a phenol group.

4-9. (canceled)

10. The medical composition of claim 1, further comprising gelatin, optionally wherein said gelatin comprises phenol-modified gelatin.

11. (canceled)

12. The medical composition of claim 10, wherein said phenol-enriched synthetic polymer and said phenol-modified gelatin are present in amounts such that a ratio of phenol groups of said phenol-enriched synthetic polymer and phenol groups of said phenol-modified gelatin is about 1:2.

13. The medical composition of claim 10, wherein 20% to 80% of the total phenol in the composition is provided by said phenol-enriched synthetic polymer.

14. The medical composition of claim 1, wherein said phenol-enriched synthetic polymer has a molecular mass of 2 kDa to 10 kDa.

15. (canceled)

16. A method of crosslinking a synthetic polymer, the method comprising:
irradiating a composition comprising a photoactivatable catalyst, an electron acceptor, and a phenol-enriched synthetic polymer to initiate a cross-linking reaction.

17. The method of claim 16, wherein said irradiating comprises exposing the composition to visible light for a duration sufficient to initiate a cross-linking reaction.

18-19. (canceled)

20. The method of claim 16, wherein the synthetic polymer comprises polyethylene glycol, polyvinyl alcohol, polylactic acid, polyglycolic acid, poly(lactic-co-glycolic acid), and/or poly(glycerol sebacate).

21. The method of claim 20, wherein the synthetic polymer is a multi-arm synthetic polymer which has been chemically modified so that each arm terminates in a phenol group.

22-27. (canceled)

28. The method of claim 16, wherein the composition further comprising gelatin, optionally wherein the gelatin comprises phenol-modified gelatin.

29. (canceled)

30. The method of claim 28, wherein the phenol-enriched synthetic polymer and the phenol-modified gelatin are present in amounts such that a ratio of phenol groups of the phenol-enriched synthetic polymer and phenol groups of the phenol-modified gelatin is about 1:2.

31. The method of claim 28, wherein 20% to 80% of the total phenol in the composition is provided by the phenol-enriched synthetic polymer.

32. The method of claim 16, wherein the phenol-enriched synthetic polymer has a molecular mass of 2 kDa to 10 kDa.

33. (canceled)

34. The method of claim 16, wherein the composition is carried by a substrate suitable for implantation.

35. The method of claim 34, wherein the substrate comprises an extracellular matrix material.

36. A medical implant comprising:
a substrate suitable for implantation;
a medical adhesive carried by said substrate, said medical adhesive comprising:
a photoactivatable catalyst,
an electron acceptor, and
a phenol-enriched synthetic polymer.

37. The medical implant of claim 36, wherein said substrate comprises an extracellular matrix material, optionally wherein said extracellular matrix material comprises submucosa, renal capsule membrane, dermal collagen, dura mater, pericardium, fascia lata, serosa, peritoneum or basement membrane layers, or including liver basement membrane.

38. (canceled)

39. The medical implant of claim 37, wherein the extracellular matrix material is in sheet form, and wherein said medical adhesive is carried on a first surface of the sheet form extracellular matrix material.

40. The medical implant of claim 36, wherein the medical adhesive is a crosslinked hydrogel.

41. (canceled)

42. The medical implant of claim 36, wherein said synthetic polymer comprise polyethylene glycol, optionally wherein said polyethylene glycol comprises a multi-arm polyethylene glycol which has been chemically modified so that each arm terminates in a phenol group.

43-48. (canceled)

49. The medical implant of claim 36, further comprising gelatin, optionally wherein said gelatin comprises phenol-modified gelatin.

50. (canceled)

51. The medical implant of claim 49, wherein said phenol-enriched synthetic polymer and said phenol-modified gelatin are present in amounts such that a ratio of phenol groups of said phenol-enriched synthetic polymer and phenol groups of said phenol-modified gelatin is about 1:2.

52. The medical implant of claim 49, wherein 20% to 80% of the total phenol in the composition is provided by said phenol-enriched synthetic polymer.

53. The medical implant of claim 36, wherein said phenol-enriched synthetic polymer has a molecular mass of 2 kDa to 10 kDa.

54-69. (canceled)

70. A method of joining and/or sealing tissues in a surgical procedure, the method comprising:
applying a medical composition as described in claim 1 to a tissue portion; and
irradiating the tissue medical composition to initiate a cross-linking reaction between one or more endogenous proteins and the phenol-enriched synthetic polymer to seal the tissue portion or join the tissue portion to an adjacent tissue portion.
